(19) United States
(12) Patent Application Publication
(10) Pub. No.: US 2009/0234195 A1
(43) Pub. Date: Sep. 17, 2009

(54) Minimal incision surgical light source module with light-emitting diodes

(75) Inventors: Hon-Lun Chen, Taipei (TW); Jyh-Way Wu, Taipei (TW); Kuo-Hsin Teng, Taipei (TW)

Correspondence Address: Juan Carlos A. Marquez;c/o Stites & Harbison PLLC, 1199 North Fairfax Street, Suite 900, Alexandria, VA, 22314-1437, US

(73) Assignee: Prodisc Technology Inc.

(21) Appl. No.: 12/289,783

(22) Filed: Nov. 4, 2008

(30) Foreign Application Priority Data
Mar. 14, 2008 (TW) 097108982
Jun. 25, 2008 (TW) 097123807

(51) Int. Cl. A61B 1/07

(52) U.S. Cl. 600/249

(57) ABSTRACT

Disclosed is a minimal incision surgical light source module with a light-emitting diode (LED), comprising a first LED, a first lens set, a reflector set, a second lens set and a fiber-optic connector. The first lens set, the reflector set, the second lens set and the fiber-optic connector are sequentially arranged along a light-emitting path of the first LED in a light-coupling configuration, so that light rays emitted from the first LED are coupled into an optical fiber by the fiber-optical connector. By implementing the minimal incision surgical light source module, the light rays emitted from the first LED are concentrated and condensed into a light beam after passing through the first lens set, the reflector set and the second lens set, thereby reducing a circumference of the concentrated light beam.

### BACKGROUND OF THE INVENTION

1. Technical Field

The present invention relates to a minimal incision surgical light source module with light-emitting diodes (LEDs), and more particularly, to a minimal incision surgical light source module with LEDs for enabling minimal incision surgical illumination.

2. Description of Related Art

A minimal incision surgical light source module, when used in combination with optic fibers, can directly detect lesions inside a patient's body in a least invasive way and transmit lesion images out of the body correctly and instantly. Among the several light sources generally used with minimal incision surgical endoscopes, LEDs have constantly improved performances and brightness and, as a cold light source, can be used for a prolonged period of time without generating high heat. Therefore, LEDs have been viewed as a new alternative minimal incision surgical light source that will take the place of halogen lamps.

However, as the length of optical fibers used with a minimal incision surgical endoscope increases, light rays transmitted within the optical fibers are subject to loss, so that light-emitting efficiency decreases as the length of the optical fibers increases. Consequently, light-emitting intensity at the ends of the optical fibers is insufficient for illumination. In order to provide sufficient brightness, a plurality of LEDs must be used.

When the number of LEDs in a light source module of a minimal incision surgical endoscope is increased, the light source module has a larger volume, which not only leads to a higher cost of the minimal incision surgical endoscope, but also limits the overall volume of the minimal incision surgical endoscope. Hence, if the illumination effect of a light source module of the minimal incision surgical endoscope can be enhanced without increasing the size of the light source module, the limitation on the application length of optical fibers can be relaxed.

For instance, U.S. Pat. No. 6,832,849 discloses a “light radiation device, light source device, light radiation unit, and light connection mechanism”, in which the light source device is configured to provide a light source in the form of a light beam and comprises a light-guiding member assembly and a casing. Therein, the light-guiding member assembly includes a plurality of bundled optical fibers or a glass rod and one or a plurality of LEDs, while the casing houses a first light source lens and a second light source lens. The first light source lens collimates light rays emitted from the LED or plurality of LEDs in the light-guiding member assembly into generally parallel light rays. The second light source lens condenses the light rays from the first light source lens and introduces the condensed light rays to the light-guiding member assembly.

According to the prior art cited above, two light source lenses are arranged in the light-emitting path of each LED, so as to condense the light rays emitted from all the LEDs onto an optical coupling surface of an optical fiber. However, when there are a plurality of LEDs in the light source module, the distance from each LED to the optical coupling surface of the optical fiber is different, but the same lens combination is used in the light-emitting path of each LED. Thus, light rays emitted from some of the LEDs may be used ineffectively, thereby lowering a light-emitting efficiency of the entire light source module.

### BRIEF SUMMARY OF THE INVENTION

It is an objective of the present invention to provide a minimal incision surgical light source module with LEDs, wherein the LEDs are used as a minimal incision surgical light source and a fiber-optic connector is configured to couple the light source into an optical fiber. In addition, a lens set and a reflector set are provided for concentrating light rays emitted from each of the LEDs, so as to increase the efficiency with which the light rays are coupled into the optical fiber, thereby reducing the number of LEDs used. Therefore, sufficient illumination intensity can be achieved without increasing the volume of the minimal incision surgical light source module.

In order to increase a light source intensity for a minimal incision surgical endoscope, it is another objective of the present invention to provide a minimal incision surgical light source module with LEDs which comprises a fiber-optic connector and a light-emitting unit, both housed in a housing. The fiber-optic connector is configured to couple light rays emitted from the light-emitting unit into an optical fiber, so as to provide a minimal incision surgical light source.

It is still another objective of the present invention to provide a light-emitting unit for use with a minimal incision surgical light source module with LEDs, wherein the light-emitting unit comprises a thermally conductive base and at least one LED. Therein, the thermally conductive base can be designed with different inclination angles so that a light field generated by the at least one LED is concentratedly projected to the same area, thereby optimizing an LED light source intensity for a minimal incision surgical endoscope. Thus, by increasing the light source intensity for the minimal incision surgical endoscope, a clearer diagnostic image can be obtained.

To achieve these ends, the present invention provides a minimal incision surgical light source module with LEDs, comprising: at least two first LEDs; at least two first lens sets, each arranged in a light-emitting path of a corresponding said first LED for coupling light therefrom in a one-to-one manner; at least two reflector sets, each arranged in a light-emitting path of a corresponding said first lens set for coupling light therefrom in a one-to-one manner; a second lens set arranged in light-emitting paths of the reflector sets for coupling light therefrom; and a fiber-optic connector arranged in a light-emitting path of the second lens set for coupling light therefrom.

To achieve the aforementioned ends, the present invention further provides a minimal incision surgical light source module with an LED, comprising a third LED, a fourth lens set arranged in a light-emitting path of the third LED for coupling light therefrom, and a fiber-optic connector arranged in a light-emitting path of the fourth lens set for coupling light therefrom.

To achieve the aforementioned ends, the present invention also provides a minimal incision surgical light source module with an LED, comprising a fourth LED, a fifth lens attached to the fourth LED, and a fiber-optic connector arranged in a light-emitting path of the fifth lens for coupling light therefrom.

To achieve the aforementioned ends, the present invention further provides a minimal incision surgical light source module with an LED, comprising: a housing having a first side surface and a second side surface; a fiber-optic connector disposed on the first side surface and having an optical coupling surface; and a light-emitting unit disposed on an inner side of the second side surface and comprising a thermally conductive base and at least one LED, which is connected to the thermally conductive base in a thermally conductive manner and has a light field projecting to the optical coupling surface.

To achieve the aforementioned ends, the present invention also provides a light-emitting unit for use with a minimal incision surgical light source module with LEDs, wherein the light-emitting unit comprises a thermally conductive base and at least one LED, which is connected to the thermally conductive base in a thermally conductive manner and has a light field projecting to an optical coupling surface of a fiber-optic connector.

The present invention can be implemented to provide at least the following advantageous effects:
1. The lens sets are arranged to condense the light rays emitted from the LEDs, thereby improving the light utilization efficiency of the LEDs;
2. As the light utilization efficiency of the LEDs is improved, the number of LEDs used can be reduced, thereby allowing the minimal incision surgical light source module to be miniaturized; and
3. As the lens sets condense the light rays emitted from the LEDs, light coupled into the optical fiber can have a low loss rate, thereby enhancing a light-emitting intensity of the minimal incision surgical light source.

A detailed description of further features and advantages of the present invention is given below, so that a person skilled in the art is allowed to understand and carry out the technical content of the present invention, and can readily comprehend the objectives and advantages of the present invention by reviewing the content disclosed herein, the appended claims and the accompanying drawings.

### BRIEF DESCRIPTION OF THE SEVERAL VIEWS OF THE DRAWINGS

The invention as well as a preferred mode of use, further objectives and advantages thereof will be best understood by reference to the following detailed description of illustrative embodiments when read in conjunction with the accompanying drawings, wherein:

FIG. 1 is a perspective view of a minimal incision surgical light source module with LEDs according to a first embodiment of the present invention when assembled;

FIG. 2 is a cross-sectional view of the minimal incision surgical light source module with LEDs according to the first embodiment of the present invention;

FIG. 3 is an exploded perspective view showing another aspect of the minimal incision surgical light source module with LEDs according the first embodiment of the present invention;

FIG. 4 is a cross-sectional view showing yet another aspect of the minimal incision surgical light source module with LEDs according to the first embodiment of the present invention;

FIG. 5 is a cross-sectional view of a minimal incision surgical light source module with an LED according to a second embodiment of the present invention;

FIG. 6 shows a first embodiment of a lens set according to the present invention;

FIG. 7 shows a second embodiment of the lens set according to the present invention;

FIG. 8 shows a third embodiment of the lens set according to the present invention;

FIG. 9 shows a fourth embodiment of the lens set according to the present invention;

FIG. 10 shows a fifth embodiment of the lens set according to the present invention;

FIG. 11 is a cross-sectional view of a minimal incision surgical light source module according to a third embodiment of the present invention;

FIG. 12 a perspective view of a minimal incision surgical light source module according to a fourth embodiment of the present invention when assembled;

FIG. 13 is across-sectional view of the minimal incision surgical light source module according to the fourth embodiment of the present invention;

FIG. 14 is a cross-sectional view showing another aspect of the minimal incision surgical light source module according to the fourth embodiment of the present invention;

FIG. 15 is a perspective view of a light-emitting unit of the minimal incision surgical light source module according to the fourth embodiment of the present invention;

FIG. 16 is a cross-sectional view showing still another aspect of the minimal incision surgical light source module according to the fourth embodiment of the present invention; and

FIG. 17 is a perspective view showing another aspect of the light-emitting unit of the minimal incision surgical light source module according to the fourth embodiment of the present invention.

### DETAILED DESCRIPTION OF THE INVENTION

### First Embodiment

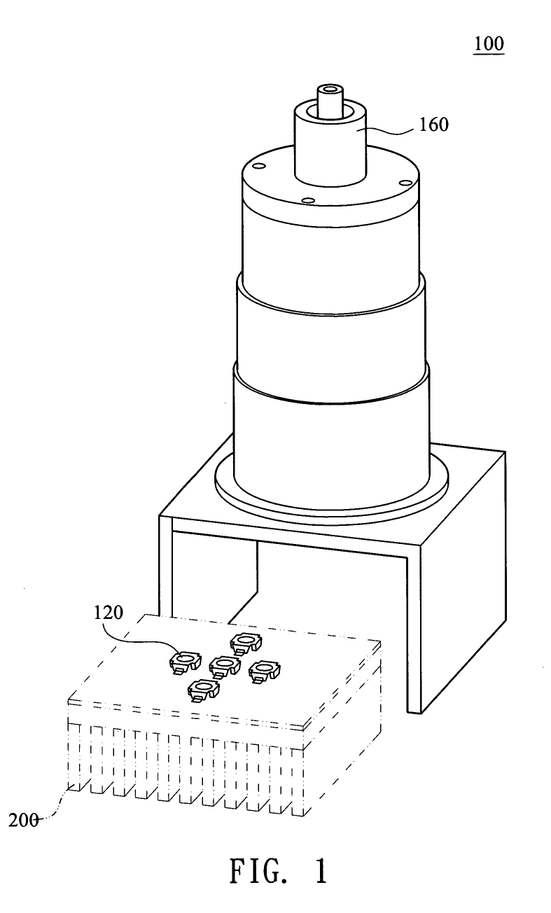

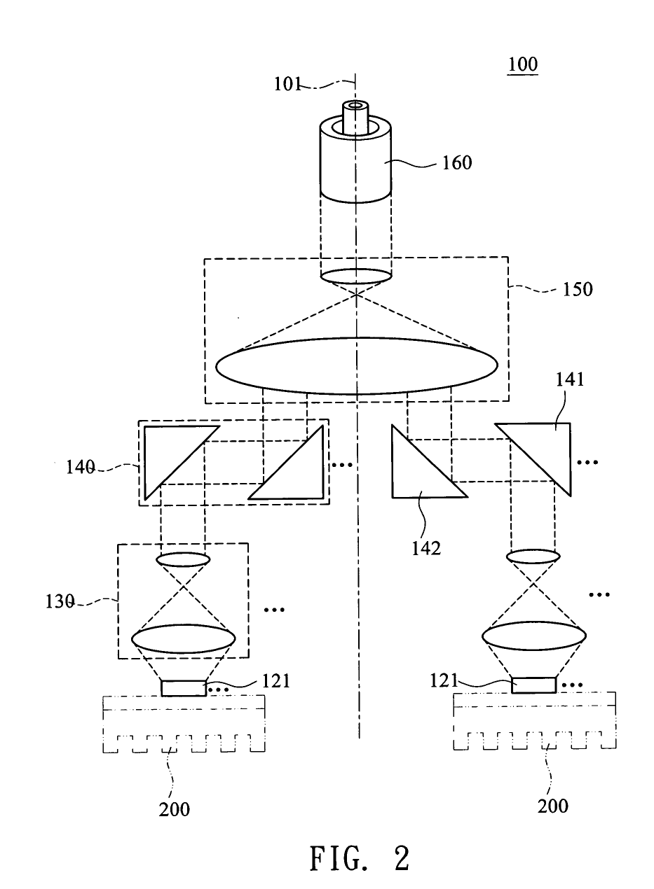

As shown in FIGS. 1 and 2, a minimal incision surgical light source module 100 with LEDs according to this embodiment of the present invention comprises at least two first LEDs 121, at least two first lens sets 130, at least two reflector sets 140, a second lens set 150, and a fiber-optic connector 160.

The first LEDs 121 serve as a light source of a minimal incision surgical endoscope, wherein each of the first LEDs 121 can be connected to a heat dissipation device 200 in a thermally conductive manner to facilitate heat dissipation from each of the first LEDs 121. Alternatively, a plurality of LEDs 120 can be used as the light source and be configured so as to be replaceable, so that any one of the LEDs 120 can be conveniently replaced when damaged.

Each of the first lens sets 130 is arranged in a light-emitting path of a corresponding said first LED 121 for coupling light therefrom in a one-to-one manner. Light rays emitted from the first LEDs 121 have light-emitting paths passing through the first lens sets 130.

Each of the reflector sets 140 is arranged in a light-emitting path of a corresponding said first lens set 130 for coupling light therefrom in a one-to-one manner. Each of the reflector sets 140 comprises a first reflector unit 141 and a second reflector unit 142, wherein the first and second reflector units 141 and 142 are arranged relative to each other in a light-coupling configuration.

Light rays emitted from the first lens sets 130 fall on the first reflector units 141 and are reflected for the first time by the first reflector units 141. As a result, light-emitting paths of the light rays have their directions changed. The light rays reflected by the first reflector units 141 are projected to the second reflector units 142, which are arranged relative to the first reflector units 141 in a light-coupling configuration, and reflected for a second time by the second reflector units 142, so that the directions of the light-emitting paths of the light rays are changed again.

The reflector sets 140 are provided to change the directions of light-emitting paths. Therefore, light rays coming from each of the first lens sets 130 can have the directions of their light-emitting paths changed by a corresponding said reflector set 140, so that all the light-emitting paths are concentrated toward an axis 101 of the minimal incision surgical light source module 100, thereby condensing the light again.

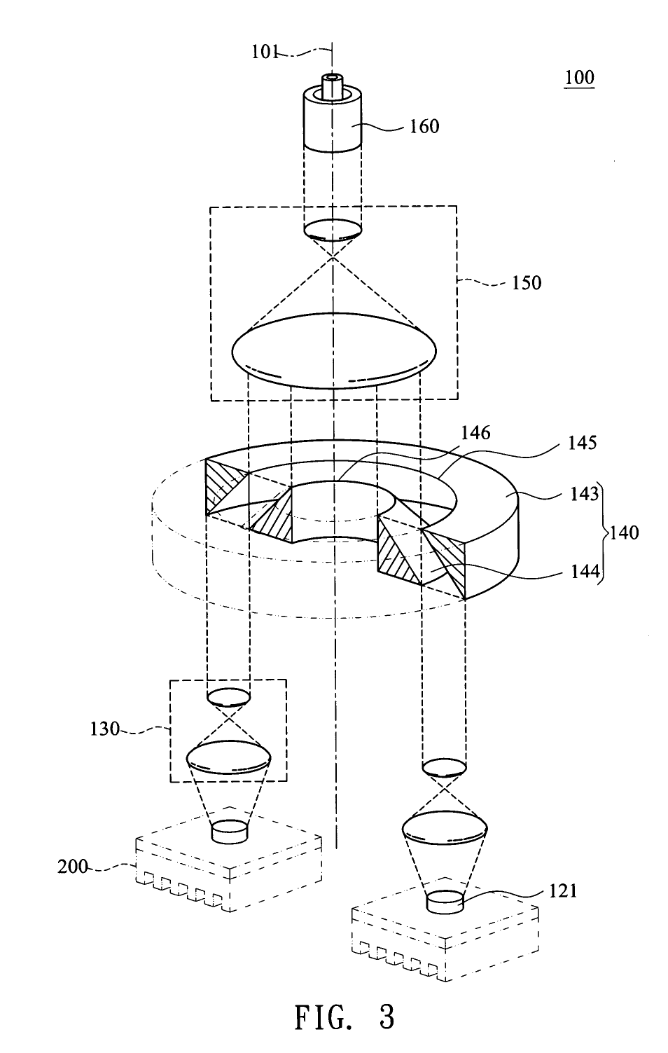

Referring to FIG. 3, in addition to being arranged in the light-emitting paths of the first lens sets 130 in a one-to-one light-coupling configuration, the first and second reflector units 141 and 142 may also be integrally formed as a first annular reflector 143 and a second annular reflector 144, respectively, wherein the first annular reflector 143 is arranged concentrically with respective to and outside the second annular reflector 144.

The first annular reflector 143 formed by the first reflector unit 141 is located at a first concentric circle 145 having a center defined by the axis 101 of the light source module, while the second annular reflector 144 formed by the second reflector unit 142 is located at a second concentric circle 146 and disposed relative to the first annular reflector 143 in such a way as to couple light therefrom. By substituting the integrally formed first and second annular reflectors 143 and 144 for the first and second reflector units 141 and 142 which are arranged relative to each other in a one-to-one configuration, assembly and production steps of the minimal incision surgical light source module 100 can be simplified.

The second lens set 150 is arranged in light-emitting paths of the reflector sets 140 for coupling light therefrom. A light beam projecting into the second lens set 150 along the light-emitting path of each of the reflector sets 140 also passes through the second lens set 150.

The fiber-optic connector 160 is arranged in a light-emitting path of the second lens set 150 for coupling light therefrom, so that light rays emitted from the first LEDs 121 can be coupled into an optical fiber by the fiber-optic connector 160 after passing through the first lens sets 130, the reflector sets 140, the second lens set 150 and the fiber-optic connector 160, thereby providing illumination for minimal incision surgery.

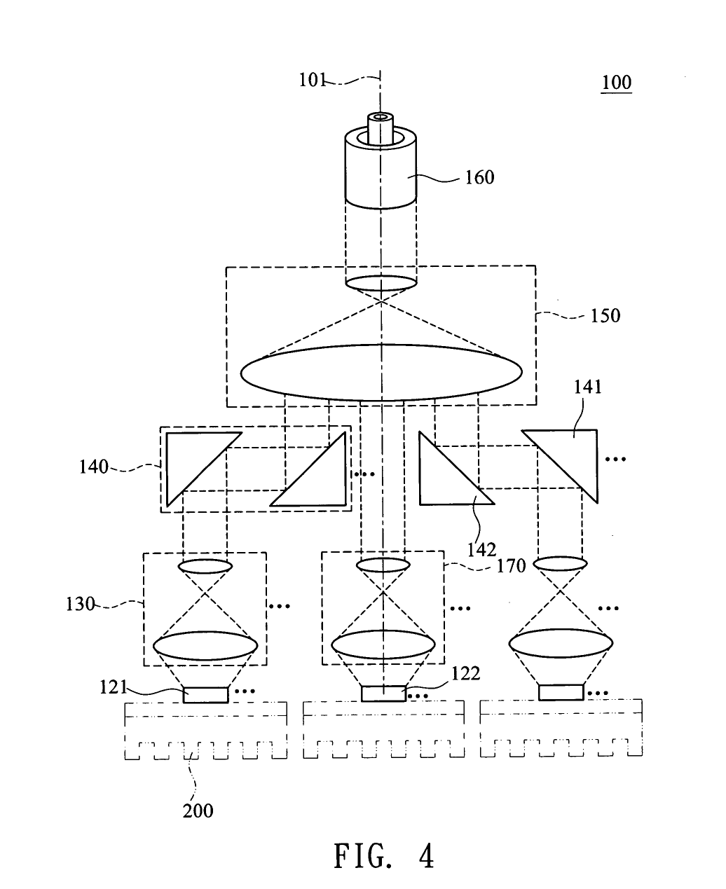

As shown in FIG. 4, the minimal incision surgical light source module 100 with LEDs may further comprise a third lens set 170, which is arranged on an axis of the second lens set 150 and in a light-emitting path of a second LED 122. The second LED 122 and the third lens set 170 are arranged sequentially along the axis of the second lens set 150, so that the light-emitting path of the second LED 122 passes through the third lens set 170.

### Second Embodiment

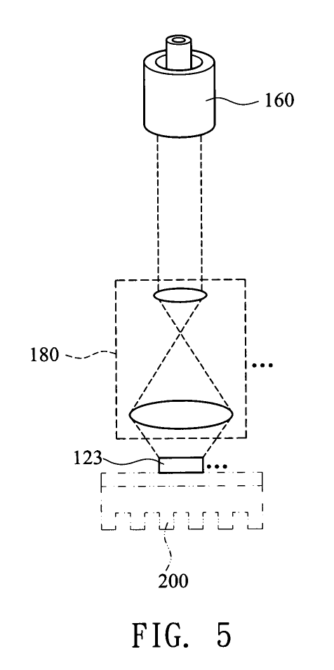

As shown in FIG. 5, a minimal incision surgical light source module 100 with an LED according to this embodiment comprises a third LED 123, a fourth lens set 180, and a fiber-optic connector 160.

The third LED 123 serves as a light source of a minimal incision surgical endoscope and can be connected to a heat dissipation device 200 in a thermally conductive manner to facilitate heat dissipation from the third LED 123.

The fourth lens set 180 is arranged in a light-emitting path of the third LED 123 for coupling light therefrom. Light rays emitted from the third LED 123 have light-emitting paths passing through the fourth lens set 180.

The fiber-optic connector 160 is arranged in a light-emitting path of the fourth lens set 180 for coupling light therefrom. The fiber-optic connector 160 allows the light rays emitted from the third LED 123 to be coupled into an optical fiber after the light rays pass through the fourth lens set 180 and the fiber-optic connector 160, thereby providing illumination for minimal incision surgery.

The first lens sets 130, the second lens set 150, the third lens set 170 and the fourth lens set 180 in the first embodiment and the second embodiment can each comprise a plurality of convex lenses 131, or at least one convex lens 131 and at least one concave lens 132 arranged in a particular way.

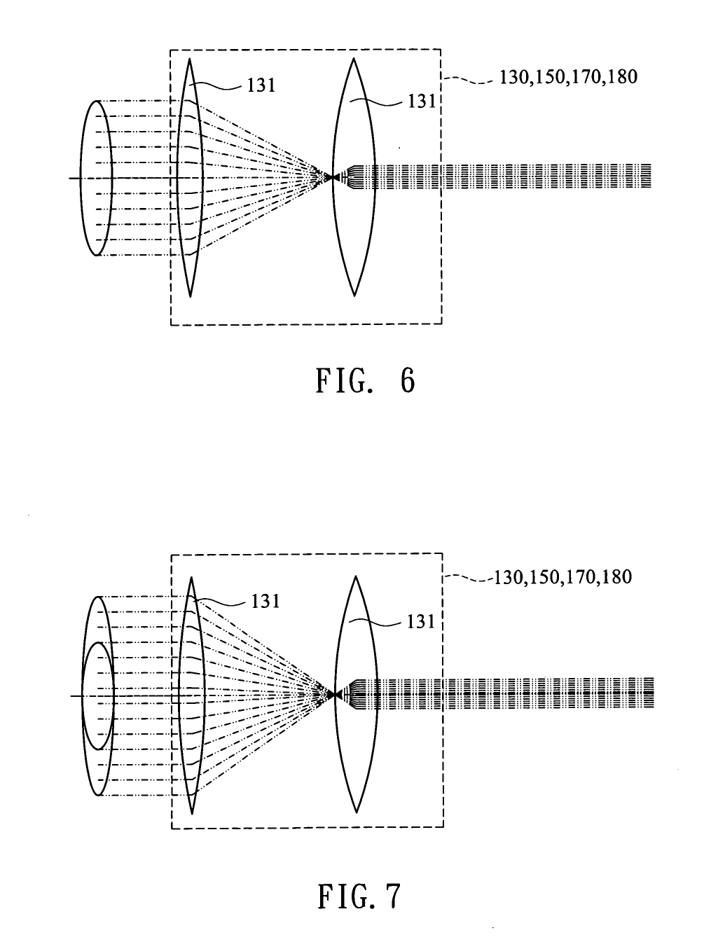

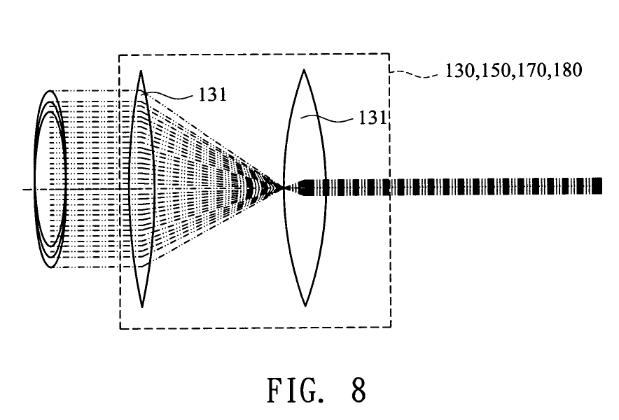

As shown in FIGS. 6, 7 and 8, the first lens sets 130, the second lens set 150, the third lens set 170 or the fourth lens set 180 can each comprise a plurality of, such as two, convex lenses 131 arranged in a particular way. The convex lenses 131, owing to their light-converging effect, can condense light rays emitted from the light source module. When the light source module comprises a plurality of, such as two or three, LEDs 120, light rays emitted from the plurality of LEDs 120 can pass through a corresponding said first lens set 130, the second lens set 150, the third lens set 170 or the fourth lens set 180, each comprising the plurality of convex lenses 131, and be condensed into a light beam.

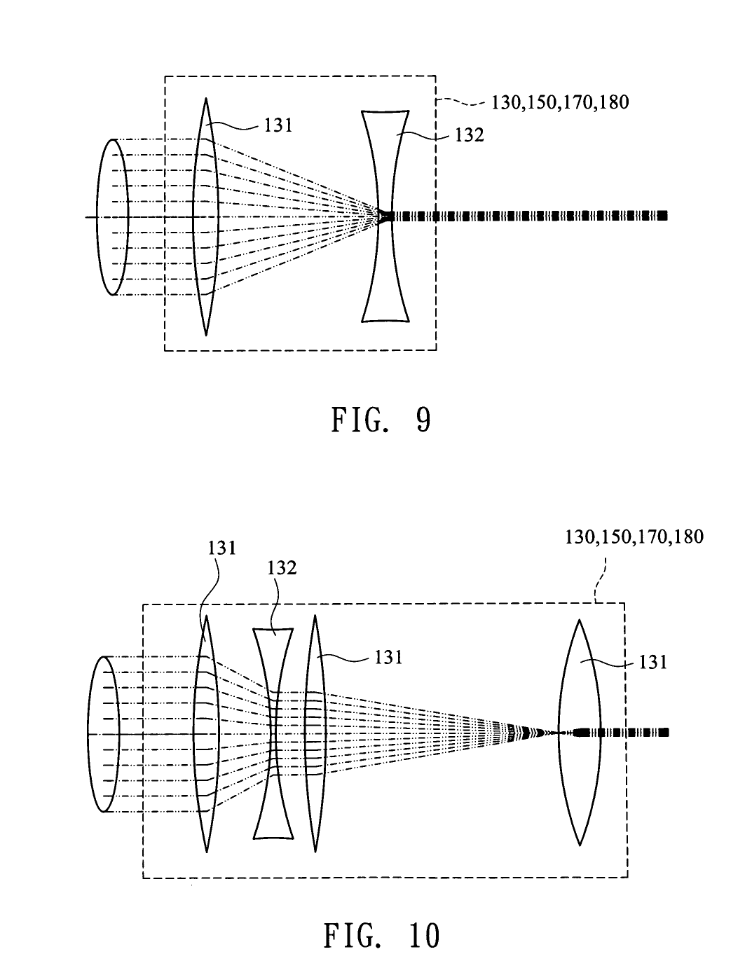

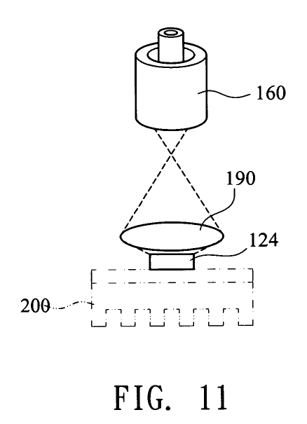

Alternatively, as shown in FIGS. 9 and 10, the first lens sets 130, the second lens set 150, the third lens set 170 or the fourth lens set 180 can each comprise at least one convex lens 131 and at least one concave lens 132, such as three convex lenses 131 and one concave lens 132, arranged in a particular way. As the convex lenses 131 and the concave lens 132 can converge and diverge light, respectively, light rays emitted from each of the LEDs 120 in the light source module can pass through a corresponding said first lens set 130, the second lens set 150, the third lens set 170 or the fourth lens set 180, each comprising the at least one concave lens 132 and the at least one convex lenses 131, and be condensed into a light beam.

Therefore, by virtue of the plurality of convex lenses 131, or the at least one convex lens 131 and at least one concave lens 132, in the first lens sets 130, the second lens set 150, the third lens set 170 or the fourth lens set 180, the light rays emitted from each of the LEDs 120 will be condensed so as to provide a light-converging/condensing effect. Thus, the light rays become more concentrated, thereby enhancing the light-emitting intensity of the minimal incision surgical light source module 100.

### Third Embodiment

As shown in FIG. 11, a minimal incision surgical light source module 100 with an LED according to this embodiment comprises a fourth LED 124, a fifth lens 190, and a fiber-optic connector 160.

The fourth LED 124 serves a light source of a minimal incision surgical endoscope and can be connected with a heat dissipation device 200 in a thermally conductive manner to facilitate heat dissipation from the fourth LED 124.

The fifth lens 190 is attached to the fourth LED 124. More particularly, the fifth lens 190 is fixedly attached to a light-emitting surface of the fourth LED 124 by an optical glue. The fifth lens 190 can be a convex lens 131, so that, through a light-converging effect of the convex lens 131, light rays emitted from the fourth LED 124 can be projected concentratedly to an optical coupling surface of the fiber-optic connector 160.

The fiber-optic connector 160 is arranged in a light-emitting path of the fifth lens 190 for coupling light therefrom. With the fiber-optic connector 160, the light rays emitted from the fourth LED 124 and passing through the fifth lens 190 and the fiber-optic connector 160 can be coupled into an optical fiber and serve as an illumination light source for minimal incision surgery.

### Fourth Embodiment

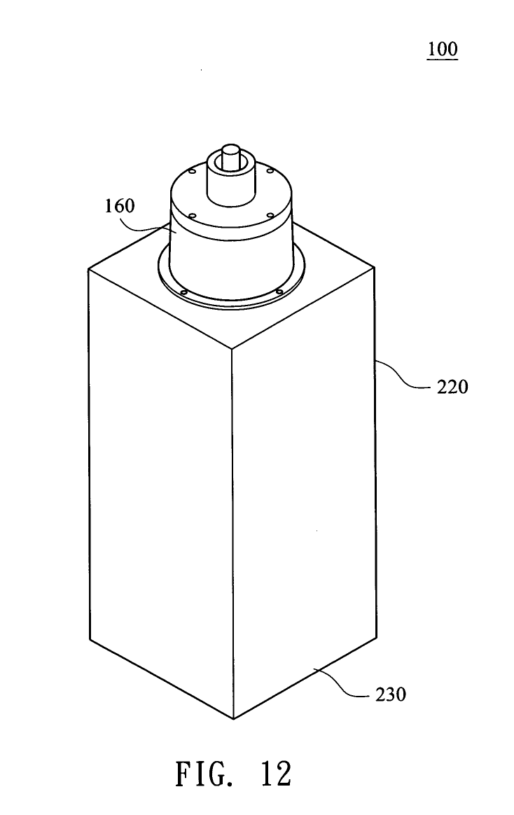

Referring to FIG. 12, a minimal incision surgical light source module 100 according to this embodiment comprises a housing 220, a fiber-optic connector 160 and a light-emitting unit 230.

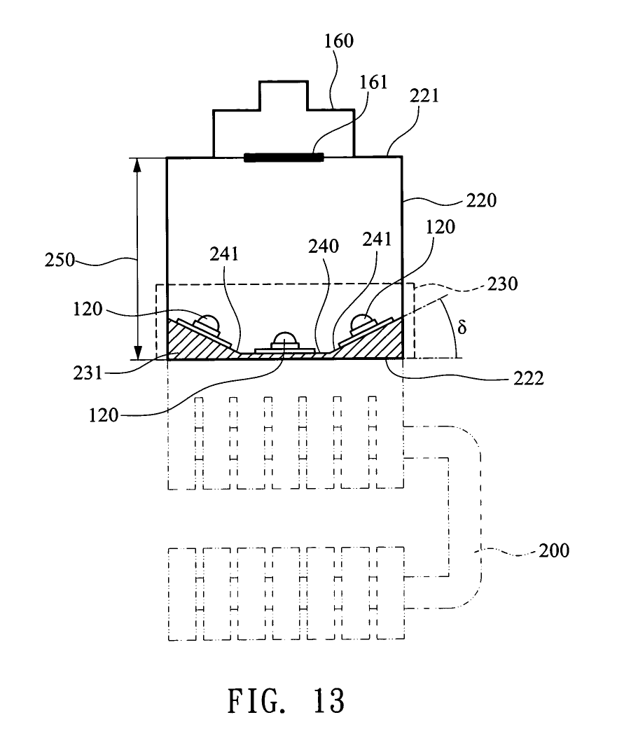

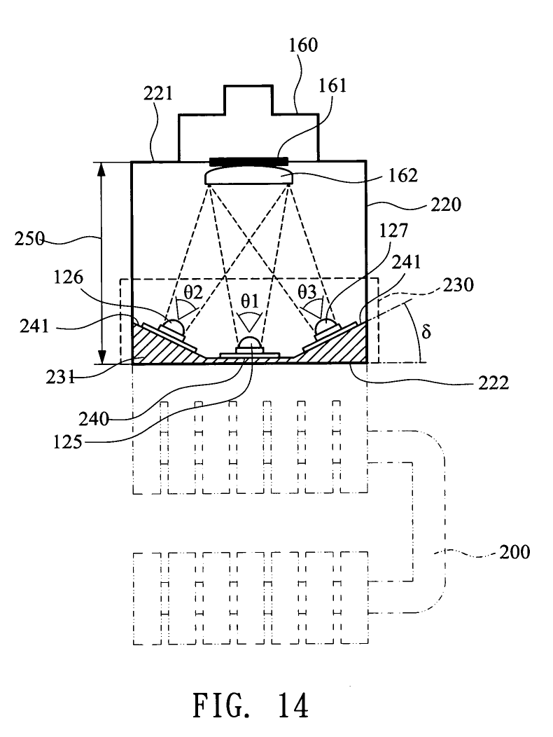

As shown in FIG. 13, the housing 220 has a first side surface 221 and a second side surface 222, wherein the first side surface 221 is used to secure the fiber-optic connector 160 and protect an inner structure of the minimal incision surgical light source module 100, while the second side surface 222 is located opposite the first side surface 221 for securing the light-emitting unit 230.

The fiber-optic connector 160 is disposed on the first side surface 221 for connecting with an optical fiber. In addition, the fiber-optic connector 160 has an optical coupling surface 161 for coupling light rays emitted from the light-emitting unit 230 to the fiber-optic connector 160, so as to provide illumination for minimal incision surgery. A vertical distance between the optical coupling surface 161 and the second side surface 222 is defined as a first distance 250, which can be changed to adjust a projection distance between the light-emitting unit 230 and the optical coupling surface 161.

The light-emitting unit 230 is disposed on an inner side of the second side surface 222 of the housing 220 and comprises a thermally conductive base 231 and at least one LED 120. The thermally conductive base 231 is disposed on the inner side of the second side surface 222 and can have a level surface 240 and at least one pair of symmetric inclined surfaces 241 for securing the LED 120 and facilitating thermal conduction from the LED 120. When the thermally conductive base 231 has one said pair of symmetric inclined surfaces 241, each having an inclination angle δ, the inclination angles δ can be designed to control a projection direction of the LED 120. The thermally conductive base 231 can be further connected with a heat dissipation device 200 in a thermally conductive manner to facilitate heat dissipation from the LED 120.

The LED 120 is connected to the thermally conductive base 231 in a thermally conductive manner and has a light field projecting to the optical coupling surface 161. When more than one said LED 120 is used as the light source, the inclination angles δ of the thermally conductive base 231 can be changed to adjust an overall projection light field of the LEDs 120. As the LEDs 120 may have different beam angles, when the inclination angles δ of the thermally conductive base 231 are changed, it is preferable to select the LEDs 120 having the most appropriate beam angles based on the inclination angles δ, so that the light field of each said LED 120 is projected to the optical coupling surface 161, thereby increasing a minimal incision surgical light source intensity.

In addition, the light-emitting unit 230 of the minimal incision surgical light source module 100 can also comprise a plurality of LEDs 120 arranged in different ways to match different said inclination angles δ, so that the light fields of the LEDs 120 are concentratedly projected to the optical coupling surface 161, thereby increasing the minimal incision surgical light source intensity. More aspects of the aforementioned minimal incision surgical light source module 100 are described as follows.

FIG. 14 is a cross-sectional view showing a second aspect of the minimal incision surgical light source module 100 according to the fourth embodiment of the present invention, wherein the thermally conductive base 231 has a level surface 240 and a pair of symmetric inclined surfaces 241, while the at least one LED 120 comprises a fifth LED 125, a sixth LED 126 and a seventh LED 127. The fifth LED 125 is connected to the level surface 240 in a thermally conductive manner while the sixth and the seventh LEDs 126 and 127 are connected respectively to the symmetric inclined surfaces 241 in the thermally conductive manner.

The fifth LED 125 has a first beam angle θ₁, the sixth LED 126 has a second beam angle θ₂ and the seventh LED 127 has a third beam angle θ₃. By designing the first, the second and the third beam angles θ₁, θ₂ and θ₃ of the fifth, the sixth and the seventh LEDs 125, 126 and 127 to match the inclination angles δ of the thermally conductive base 231, the light source of the light-emitting unit 230 can be more concentratedly projected to the optical coupling surface 161, thereby enhancing the minimal incision surgical light source intensity.

Moreover, the fiber-optic connector 160 can be further provided with an optical lens set 162, which can be any one of the lens sets 130, 150, 170 and 180 in the first, the second and the third embodiments. The optical lens set 162 can receive the light rays emitted from the light-emitting unit 230 and change refraction angles of the incident light rays so that the light rays are condensed and more concentrated. Thus, a utilization rate of light can be enhanced when the light rays are coupled into an optical fiber because the light rays are less likely to leak therefrom.

For example, the inclination angles δ of the thermally conductive base 231 can be designed at 20°, the first distance 250 can be adjusted to 2 cm, while the first, the second and the third beam angles θ₁, θ₂ and θ₃ are all set at 60°, so that the light rays emitted from the light-emitting unit 230 are projected to the optical coupling surface 161 more concentratedly.

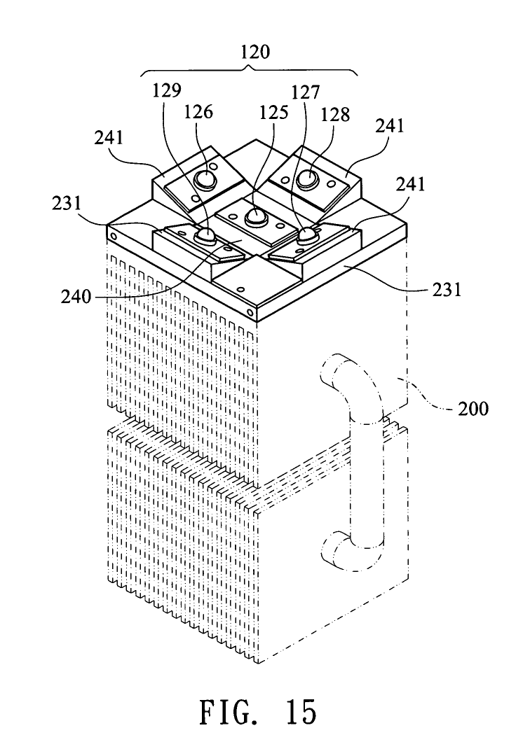

As shown in FIG. 15, the thermally conductive base 231 can also have a level surface 240 and two pairs of symmetric inclined surfaces 241 while the at least one LED 120 comprises a fifth LED 125 connected to the level surface 240 in a thermally conductive manner; and a sixth LED 126, a seventh LED 127, an eighth LED 128 and a ninth LED 129 connected respectively to the symmetric inclined surfaces 241 in the thermally conductive manner.

The fifth to the ninth LEDs 125 to 129 have a first to a fifth beam angles θ₁ to θ₅, respectively. By properly arranging the plurality of LEDs 120 and matching the beam angles thereof with the inclination angles δ of the thermally conductive base 231, the light fields of the LEDs 120 can be more concentrated, thereby enhancing the overall light source intensity of the light-emitting unit 230, so that an increased minimal incision surgical light source intensity can be achieved.

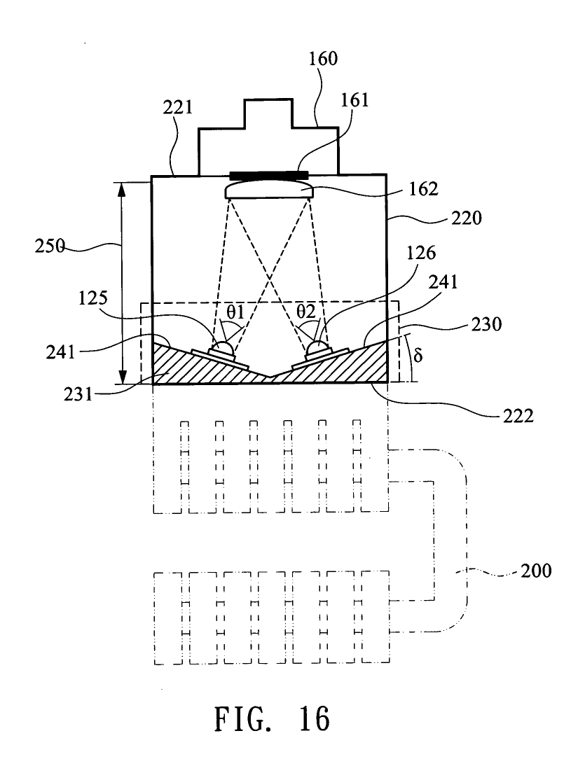

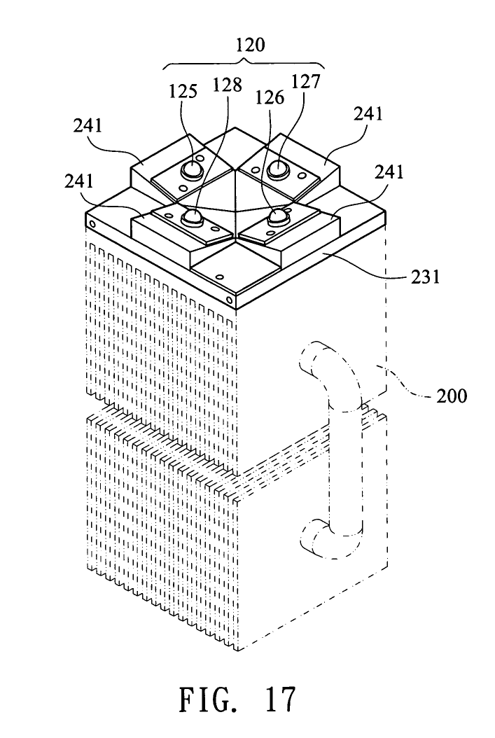

FIG. 16 illustrates a third aspect of the minimal incision surgical light source module 100 according to the fourth embodiment of the present invention, wherein the thermally conductive base 231 is not provided with the level surface 240 shown in FIG. 14 while the at least one LED 120 in the light-emitting unit 230 comprises the fifth LED 125 and the sixth LED 126, which are connected respectively to the symmetric inclined surfaces 241 of the thermally conductive base 231 in a thermally conductive manner. By matching the first and the second beam angles θ₁ and θ₂ of the fifth LED 125 and the sixth LED 126 with the inclination angles δ of the thermally conductive base 231, the light rays emitted from the light-emitting unit 230 can be projected to the optical coupling surface 161 more concentratedly before the light rays are coupled into the optical fiber by the fiber-optic connector 160, so that the minimal incision surgical light source intensity is increased.

FIG. 17 illustrates a second aspect of the light-emitting unit 230 of the minimal incision surgical light source module 100 according to the fourth embodiment of the present invention, wherein the thermally conductive base 231 is not provided with the level surface 240 in FIG. 15 while the at least one LED 120 comprises the fifth LED 125, the sixth LED 126, the seventh LED 127 and the eighth LED 128, which are connected respectively to the symmetric inclined surfaces 241 of the thermally conductive base 231 in a thermally conductive manner. By adjusting an arrangement of the LEDs 120 and designing the inclination angles δ of the thermally conductive base 231 to match the beam angles of the plurality of LEDs 120, the light rays emitted from the LEDs 120 can be more concentrated so as to increase the minimal incision surgical light source intensity.

The present invention has been described with preferred embodiments thereof, which are intended to demonstrate features of the present invention so that a person skilled in the art can readily understand and carry out the content disclosed herein, but are not intended to limit the scope of the present invention. Therefore, all equivalent changes or modifications made to the embodiments without departing from the spirit of the present invention should be encompassed by the appended claims.

1. A minimal incision surgical light source module with light-emitting diodes (LEDs), comprising:
at least two first LEDs;
at least two first lens sets, each said first lens set arranged in a light-emitting path of a corresponding said first LED for coupling light therefrom in a one-to-one manner;
at least two reflector sets, each said reflector set arranged in a light-emitting path of a corresponding said first lens set for coupling light therefrom in a one-to-one manner;
a second lens set arranged in light-emitting paths of the reflector sets for coupling light therefrom; and
a fiber-optic connector arranged in a light-emitting path of the second lens set for coupling light therefrom.

2. The minimal incision surgical light source module as claimed in claim 1, wherein each said first lens set is constructed from at least one convex lens and at least one concave lens, or is constructed from a plurality of convex lenses.

3. The minimal incision surgical light source module as claimed in claim 1, wherein each said reflector set comprises a first reflector unit and a second reflector unit arranged relative to each other in a light-coupling configuration.

4. The minimal incision surgical light source module as claimed in claim 3, wherein the first reflector unit is a first annular reflector.

5. The minimal incision surgical light source module as claimed in claim 3, wherein the second reflector unit is a second annular reflector.

6. The minimal incision surgical light source module as claimed in claim 1, wherein the second lens set is constructed from at least one convex lens and at least one concave lens.

7. The minimal incision surgical light source module as claimed in claim 1, wherein the second lens set comprises a plurality of convex lenses.

8. The minimal incision surgical light source module as claimed in claim 1, further comprising a third lens set arranged on an axis of the second lens set and in a light-emitting path of a second LED.

9. The minimal incision surgical light source module as claimed in claim 8, wherein the third lens set is constructed from at least one convex lens and at least one concave lens, or is constructed from a plurality of convex lenses.

10. A minimal incision surgical light source module with an LED, comprising:
a third LED;
a fourth lens set arranged in a light-emitting path of the third LED for coupling light therefrom; and
a fiber-optic connector arranged in a light-emitting path of the fourth lens set for coupling light therefrom.

11. The minimal incision surgical light source module as claimed in claim 10, wherein the fourth lens set is constructed from at least one convex lens and at least one concave lens, or is constructed from a plurality of convex lenses.

12. A minimal incision surgical light source module with an LED, comprising:
a fourth LED;
a fifth lens attached to the fourth LED; and
a fiber-optic connector arranged in a light-emitting path of the fifth lens for coupling light therefrom.

13. The minimal incision surgical light source module as claimed in claim 12, wherein the fifth lens is a convex lens.

14. A minimal incision surgical light source module with an LED, comprising:
a housing having a first side surface and a second side surface;
a fiber-optic connector disposed on the first side surface and having an optical coupling surface; and
a light-emitting unit disposed on an inner side of the second side surface and comprising:
a thermally conductive base; and
at least one LED connected to the thermally conductive base in a thermally conductive manner and having a light field projecting to the optical coupling surface.

15. The minimal incision surgical light source module as claimed in claim 14, wherein the fiber-optic connector further comprises an optical lens set disposed on an incident side of the optical coupling surface.

16. The minimal incision surgical light source module as claimed in claim 14, wherein the thermally conductive base has a level surface and at least one pair of symmetric inclined surfaces while the light-emitting unit comprises a fifth LED connected to the level surface in the thermally conductive manner, and a sixth LED and a seventh LED connected respectively to the symmetric inclined surfaces in the thermally conductive manner.

17. The minimal incision surgical light source module as claimed in claim 14, wherein the thermally conductive base has a level surface and at least two pairs of symmetric inclined surfaces while the light-emitting unit comprises a fifth LED connected to the level surface in the thermally conductive manner, and a sixth LED, a seventh LED, an eighth LED and a ninth LED connected respectively to the symmetric inclined surfaces in the thermally conductive manner.

18. The minimal incision surgical light source module as claimed in claim 14, wherein the thermally conductive base has at least one pair of symmetric inclined surfaces while the light-emitting unit comprises a fifth LED and a sixth LED connected respectively to the symmetric inclined surfaces in the thermally conductive manner.

19. The minimal incision surgical light source module as claimed in claim 14, wherein the thermally conductive base has at least two pairs of symmetric inclined surfaces while the light-emitting unit comprises a fifth LED, a sixth LED, a seventh LED and an eighth LED connected respectively to the symmetric inclined surfaces in the thermally conductive manner.

20. A light-emitting unit for use with a minimal incision surgical light source module with an LED, comprising:
a thermally conductive base; and
at least one LED connected to the thermally conductive base in a thermally conductive manner and having a light field projecting to an optical coupling surface of a fiber-optic connector.

21. The light-emitting unit as claimed in claim 20, wherein the thermally conductive base has a level surface and at least one pair of symmetric inclined surfaces while the LED comprises a fifth LED connected to the level surface in the thermally conductive manner, and a sixth LED and a seventh LED connected respectively to the symmetric inclined surfaces in the thermally conductive manner.

22. The light-emitting unit as claimed in claim 20, wherein the thermally conductive base has a level surface and at least two pairs of symmetric inclined surfaces while the LED comprises a fifth LED connected to the level surface in the thermally conductive manner, and a sixth LED, a seventh LED, an eighth LED and a ninth LED connected respectively to the symmetric inclined surfaces in the thermally conductive manner.

23. The light-emitting unit as claimed in claim 20, wherein the thermally conductive base has at least one pair of symmetric inclined surfaces while the LED comprises a fifth LED and a sixth LED connected respectively to the symmetric inclined surfaces in the thermally conductive manner.

24. The light-emitting unit as claimed in claim 20, wherein the thermally conductive base has at least two pairs of symmetric inclined surfaces while the LED comprises a fifth LED, a sixth LED, a seventh LED and an eighth LED connected respectively to the symmetric inclined surfaces in the thermally conductive manner.
